(19) United States
(12) Patent Application Publication
(10) Pub. No.: US 2017/0014024 A1
(43) Pub. Date: Jan. 19, 2017

(54) TELEMEDICAL THROAT EXAMINATION DEVICE

(71) Applicant: Corey Adam Wachs, Las Vegas, NV (US)

(72) Inventor: Corey Adam Wachs, Las Vegas, NV (US)

(21) Appl. No.: 14/813,489

(22) Filed: Jul. 30, 2015

Related U.S. Application Data
(60) Provisional application No. 62/191,728, filed on Jul. 13, 2015.

(51) Int. Cl. A61B 1/24; A61B 1/06

(52) U.S. Cl. CPC A61B 1/24; A61B 1/06

(57) ABSTRACT

A throat examination device, suitable for telemedical application, whereby an unskilled examiner may safely perform an examination of a patient's throat and send the results to a remotely located medically qualified personnel. The device may be used with a smartphone having a camera and comprises a viewing port, a positioning device to properly position the camera and a constrained tongue depressor to safely position the patient's tongue.

### REFERENCE TO RELATED APPLICATION

This application claims the benefit of priority to U.S. Provisional Patent Application No. 62/191728, entitled An Examination Device and Method for Telemedicine, filed on 13 Jul. 2015, which is hereby incorporated by reference, in its entirety.

### BACKGROUND

Telemedicine, the provision of medical diagnosis and treatment by a physically remote medical professional is becoming an option for medical practice. Telemedical techniques may be employed over a spectrum of scenarios. At one end of the spectrum, trained medical professionals may be present, with the patient, to safely administer diagnostic tests using instrumentation that can record and communicate with remotely located medical specialists.

At the other end of the telemedicine spectrum, the patient may be alone or, at best, have access only to untrained caregivers. This latter situation poses a significant safety risk associated with the administration of the test by unskilled personnel. In addition, the immediate availability of relatively expensive medical equipment at the patient's location is precluded.

Amongst the standard protocol for patient examination is inspection of the condition of the patient's throat. In some cases, the decision to prescribe an antibiotic or other treatment hinges, in large part, on the results of the throat examination.

The throat examination, as conventionally performed, does not lend itself to telemedicine procedures as would be performed by an unskilled individual due to the expertise required to physically perform the examination (i.e., proper and safe use of a tongue depressor) and to accurately quantify the observed results. The accurate characterization of the color of the internal surface of the throat as well as the presence of lesions or other abnormalities is normally beyond available expertise.

In addition, while providing a qualitative characterization of throat condition, conventionally performed examination does not provide quantitative results. The observed color of the throat is to some extent dependent the illumination and the color vision acuity of the examiner. The extent and other morphological features of any anomalies can be, at best, estimated. The results are to some extent subjective and may vary depending on the particular examiner. In the telemedicine environment, it is possible that different examiners having varied experience/capability profiles may perform subsequent follow-up examinations. The results of a conventional examination are not typically recorded in a manner that would facilitate quantitative comparison of the progression of the symptoms over time by the same or different examiners.

The inventor has identified the need for a device and method, suitable for telemedicine that overcomes the shortcomings of conventional approaches for diagnostic examination of the throat.

### BRIEF DESCRIPTION OF THE INVENTION

In embodiments there is disclosed a device and method for remotely performing the medical examination of the throat suitable for telemedicine as would be performed by an unskilled examiner. Paramount among the requirements for the device is safety. Examination of the throat with a tongue depressor by an untrained examiner exposes the patient to the danger of being poked or stabbed in the throat. This is especially relevant in situations where the patient is an animated child. Similar concerns exist for the case where the patient must self examine. The device therefore, in part, comprises elements that control the positioning and movement of the tongue depressor to mitigate the possibility of injury.

The extreme cost constraints imposed by the telemedicine scenario are mitigated by the adaptation of a commonly available telecommunications device, such as a smart phone, to serve as a medical diagnostic instrument. The device, to which a smart phone may be attached, satisfies the availability requirements posed by telemedicine by being sufficiently inexpensive to make widespread availability practical. As envisioned, the device could be cost-effectively distributed to all of a medical facility's patients and retained by the patient, for instance, in a medicine chest. The disposable device could be supplied in sterile packaging to be opened only when needed.

Specifically, in embodiments, there is disclosed a throat examination device that may comprise: a viewing port comprising a hollow conduit; a constrained tongue depressor hingedly attached to the hollow conduit; a positioning fixture affixed to the hollow conduit; and an imager receptacle affixed to rear of the hollow conduit. The device may further provide that the hollow conduit has a rectangular cross section and that the positioning fixture comprises a lip plate that is perpendicularly affixed to the hollow conduit and upper and lower bite pads. The lip plate may be conformal with the patient's face. The constrained tongue depressor may comprise a resilient forward bumper and may additionally be formed of a plurality of resiliently hinged sections. The device may further comprise one or more sources of illumination characterized by specified spectral properties.

In use, a smartphone, such as an iPhone™ without limitation, having a camera is placed into the receptacle of the throat examination device. The camera is aimed through the hollow conduit. The front portion of the hollow conduit is placed in the patient's mouth so that the lip plate rests against the front of the patient's mouth and the patient gently bites on the lip pads. The offset lever that is attached to the constrained tongue depressor is squeezed toward a fixed lever thereby causing the tongue depressor to push down on the tongue and provide a view of the throat. The smartphone's camera is then activated to image the throat. The image may be transmitted to a remote medical practitioner.

### DESCRIPTION OF FIGURES

The description of embodiments of the invention is facilitated by the figures.

FIG. 1 is a simplified isometric view of the device showing the viewing port, the positioning fixture, the constrained tongue depressor, and the imager receptacle.

FIG. 2 is a simplified side view of the device showing a smart phone installed in the imager receptacle.

FIG. 3 is a simplified front view of the device showing the hinge connected constrained tongue depressor.

FIG. 4 is a simplified partial upper view of the constrained tongue depressor showing a resilient forward bumper and a plurality of resiliently hinged sections.

FIG. 5 is a simplified partial lower view of the constrained tongue depressor showing resilient pads joining the hinged sections.

### DETAILED DESCRIPTION OF EMBODIMENTS OF THE INVENTION

In an embodiment, the Throat Examination Device (TED) provides for the telemedical examination of the throat. The TED comprises a viewing port, a positioning fixture, a constrained tongue depressor and an imaging device. The viewing port may be formed as a conduit, having a substantially rectangular cross section. The positioning fixture allows an unskilled examiner to properly orient and safely position the viewing port in the patient's mouth. In an embodiment, the positioning fixture may be formed as a lip plate that is perpendicularly affixed to the exterior of the conduit and is configured to control the orientation and depth of penetration of the conduit into the oral cavity. The positioning fixture further comprises cushion pads, on the upper and lower surfaces of the conduit that are configured to be gripped between the patient's upper and lower teeth or gums.

A constrained tongue depressor may be connected by means of a hinge to the lower portion of the conduit and thus connected to the positioning fixture and configured to depress the patient's tongue during the examination. The hinge connection limits the orientation and degree of freedom of movement of the constrained tongue depressor in the patient's throat. In an embodiment, the constrained tongue depressor device may incorporate additional safety elements described below.

The imaging device is attached to the rear face of the viewing port so that its field of view coincides with that of the viewing port. In a non-limiting embodiment, the imaging device may an integral component of the TED. In another non-limiting embodiment the imaging device may comprise a separable digital camera, such as a smart phone, that is attachable to the TED. In this later embodiment, the rear portion of the conduit may comprise an imager receptacle to position the smart phone's digital camera configured to image the throat through the viewing port.

In a non-limiting embodiment, the smart phone may be temporarily attached to the rear portion of the viewing port. The TED may further comprise computing equipment and a software program installed on the computing equipment that generates necessary command and control signals required by the imaging device and illumination light sources, accepts the output of the digital imager, processes the output of the digital imager, provides for the transmission of the digital image to the remote medical practitioner, and analyze the image to quantify the pertinent features. The computing equipment and software may be implemented within the smartphone.

The TED may further comprise one or more light sources configured to illuminate the portion of the throat being examined. The light sources may provide one or more specified light spectra to enhance the characterization of the condition of the throat.

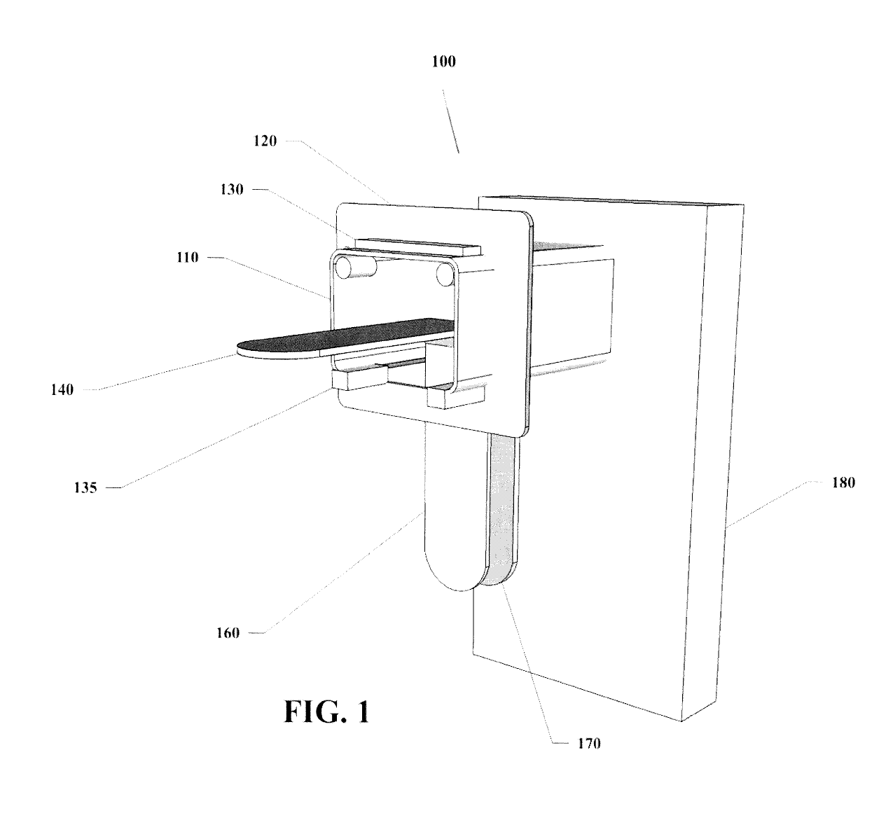

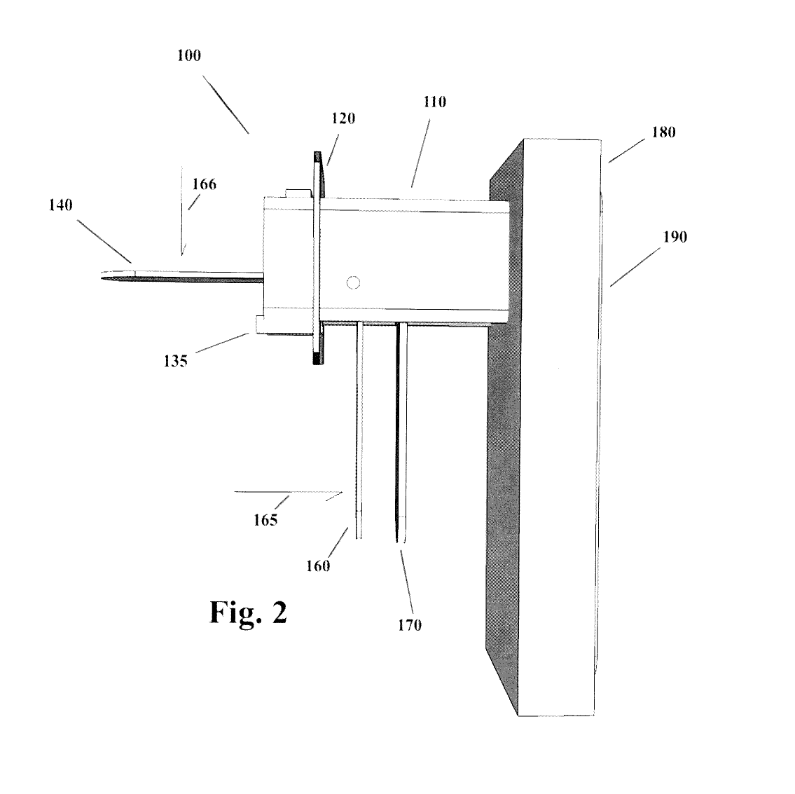

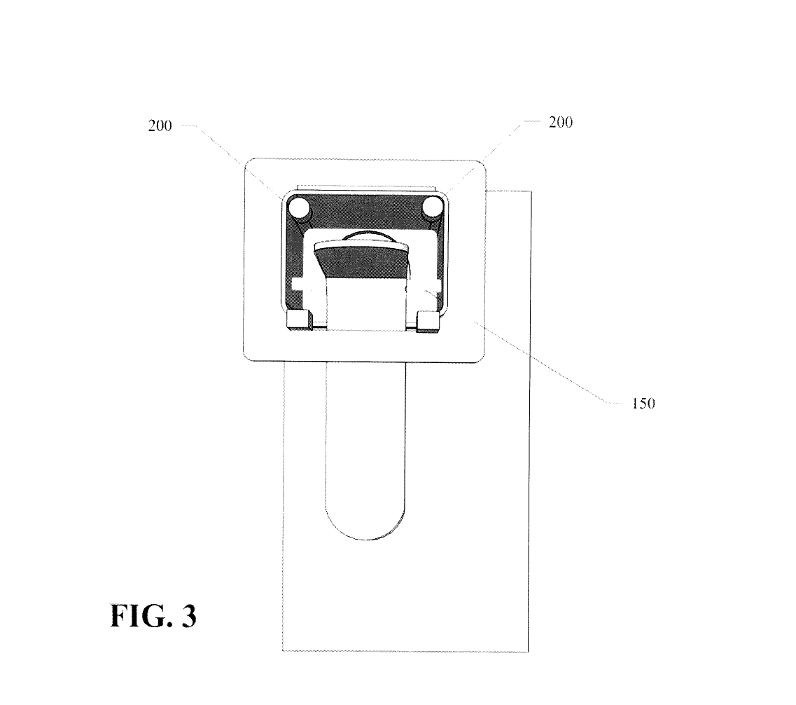

FIGS. 1 and 2 are simplified drawings of an embodiment of a throat examination device (TED) 100. A hollow conduit 110 may have, without limitation, an essentially rectangular cross-section that defines a viewing port. The cross sectional height and width of the conduit 110 is suitable for being inserted into the patient's mouth and being gripped in place by the patient's upper and lower teeth or gums. Other embodiments of the conduit 110 may have other cross sectional shapes (i.e., oval or circular, for instance, without limitation). It is anticipated that the TED 100 would be made available in various sizes to accommodate various patient's mouth sizes. A positioning fixture comprising a lip guard 120 and bite pads 130, 135 may be attached to the conduit 110. The lip guard 120 may be perpendicularly affixed to the conduit 110 to provide proper orientation and to limit the penetration of the conduit 110 into the patient's mouth and may be shaped to conform to the patient's outer lip surfaces. The forward portion of the conduit 110 may be configured to extend into the patient's mouth and may be equipped with upper pads 130 and lower pads 135 that provide a non-slip surface for contact with the teeth or gums. The lower face of the conduit 110 that extends from the forward edge of the conduit 110 to beyond the lip guard 120 is absent. A constrained tongue depressor blade 140 is attached by means of a hinge 150 (see FIG. 3) to the conduit 110 to the rear of the lower junction between the conduit and the lip guard 120. The hinge 150 limits the range of motion of the constrained tongue depressor blade 140 for safety purposes. The rear end of the constrained tongue depressor blade 140 is attached, in proximity to the hinge 150, to an offset lever 160. The offset lever 160 may be, without limitation, perpendicular to said constrained tongue depressor blade 140. A force 165 applied to the offset lever 160, in the rearward direction, causes a corresponding downward force 166 to be applied by the constrained tongue depressor blade 140. A fixed lever 170 may be fixedly attached to the bottom conduit 110 to the rear of the hinge 150. With the conduit 110 gripped between the patient's upper and lower teeth, squeezing the lower portion of the offset lever 160 against the lower portion of the fixed lever 170 would cause a downward displacement of the constrained tongue depressor blade 140 thereby depressing the patient's tongue.

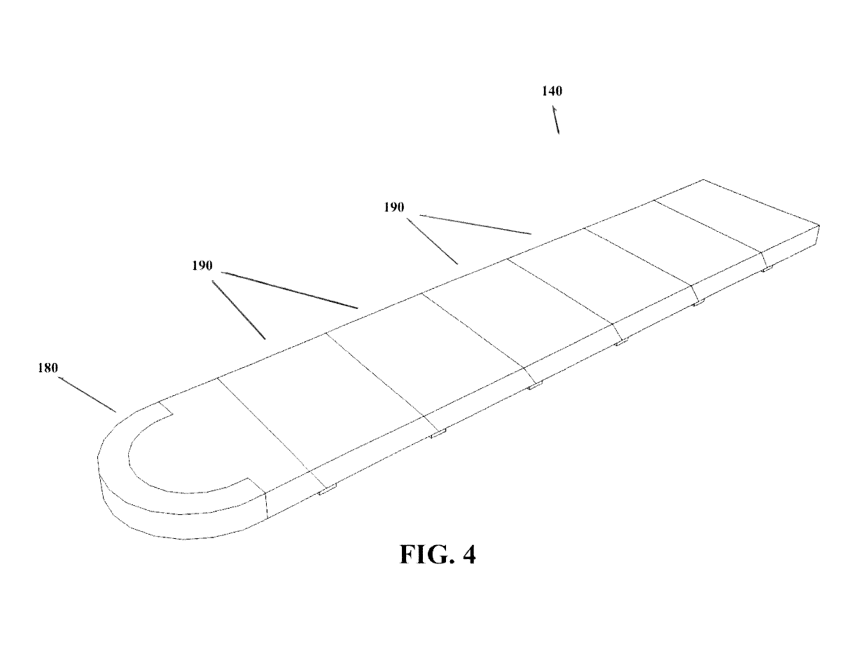

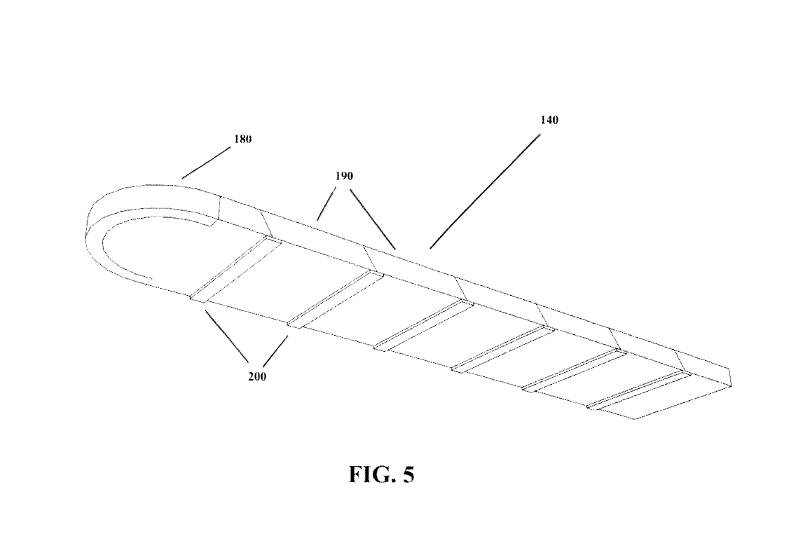

A potential safety issue is associated with the constrained tongue depressor blade 140. Insertion of the constrained tongue depressor blade 140 into the patient's mouth may result in poking or stabbing of the patient's throat. As shown in FIGS. 4 and 5, the constrained tongue depressor blade 140 may comprise one or more safety elements to mitigate these possibilities. The leading edge of the constrained tongue depressor 140 may include a forward bumper zone 180 comprising a resilient material that could cushion any potential contact force. Alternatively or in combination, the constrained tongue depressor blade 140 may be formed of a plurality of sections 190 that are joined at their bottom surfaces by pads 200 of resilient material. The junction faces 210 between the sections 190 may be angled relative to a vertical plane to enhance resistance to upward flexure. The constrained tongue depressor blade 140 will flex when a downward force is applied, such as would result from contact with the upper roof of the throat. The application of an upward force to the constrained tongue depressor blade 140, such as would occur in response to contact with the top of the tongue, would not cause flexure. This property can minimize the risk of poking or jabbing the upper or ear surfaces of the throat.

An imager receptacle 180 may be attached to the rear extremity of the conduit 110. As shown in FIG. 2, the imaging device receptacle 180 is configured to removably secure an imaging device 190 to the conduit 110 so that the field of view of the imaging device 190 corresponds to that presented by the viewing port. The imaging device receptacle 180 comprises an imaging window 220 (see FIG. 6) that is positioned so that the imaging device's lens has an un-obscured view through the viewing port. The length of the conduit 110 extending rearward from the junction with the lip guard is selected so that the depth of focus of the imaging device 190 encompasses the patient's observable throat.

In another embodiment, the imaging device receptacle 180 may be configured to attach the TED 100 to an imaging device 190 associated with a computer screen such as, without limitation, a laptop computer. Suitable imaging devices 190, for which imaging device receptacle may be made available, may comprise, without limitation, smartphones, videocams, imaging devices incorporated into computers and digital cameras.

In an embodiment, the smartphone's existing flash may be employed to illuminate the throat. In an alternative embodiment, the TED 100 may include one or more illumination sources 200 suitable for examination with the imaging device 190. The illumination sources 200 are properly baffled so that the imaging device 190 is not exposed to direct illumination. In a further embodiment, the illumination sources 200 may provide illumination having one or more specified spectral properties. For non-limiting example purposes, green illumination may provide enhanced contrast between a red throat and white blisters as compared to white light. As a further example, ultraviolet illumination may be advantageous for detecting oral mucosal abnormalities including viral, fungal and bacterial infections, inflammation from a variety of causes (lichen planus and lichenoid reactions, allergy to amalgam fillings, etc.), squamous papillomas, salivary gland tumors, cancer and pre-cancer. It is further provided that quantitative comparisons of images made under different spectra may provide a further useful diagnostic characteristic. The computer may generate command and control signals. Power for the illumination sources 200 may be self contained or may be externally provided.

While the invention has been described with respect to preferred embodiments, those skilled in the art will readily appreciate that various changes and/or modifications can be made to the invention without departing from the spirit or scope of the invention as defined by the appended claims. All documents cited herein are incorporated by reference herein in their entirety for teachings of additional or alternative details, features and/or technical background.

What is claimed is:

1. A device comprising:
a viewing port comprising a hollow conduit;
a constrained tongue depressor hingedly attached to said hollow conduit;
a positioning fixture affixed to said hollow conduit;
an imager receptacle affixed to rear of said hollow conduit.

2. The device, in accordance with claim 1, wherein:
said hollow conduit has a rectangular cross section;
said positioning fixture comprises a lip plate that is perpendicularly affixed to said hollow conduit and upper and lower bite pads.

3. The device, in accordance with claim 1, wherein:
said lip plate is conformal with the patient's face.

4. The device, in accordance with claim 1, wherein said constrained tongue depressor comprises a resilient forward bumper.

5. The device, in accordance with claim 1, wherein said constrained tongue depressor is formed of a plurality of resiliently hinged sections.

6. The device, in accordance with claim 1, wherein said constrained tongue depressor is attached to an offset lever.

7. The device, in accordance with claim 1, further comprising one or more sources of illumination.

8. The device, in accordance with claim 7, wherein said sources of illumination emit illumination having one or more specified spectral characteristics.
